(19) United States
(12) Patent Application Publication
(10) Pub. No.: US 2001/0007859 A1
(43) Pub. Date: Jul. 12, 2001

(54) PREGNANE GLUCURONIDES

(76) Inventors: CHRISTOPHER P. MILLER, WAYNE, PA (US); BACH D. TRAN, BALTIMORE, MD (US); MICHAEL D. COLLINI, CLIFTON HEIGHTS, PA (US)

Correspondence Address: AMERICAN HOME PRODUCTS CORPORATION, PATENT SECTION, FIVE GIRALDA FARMS, MADISON, NJ, 07940-0874, US

(21) Appl. No.: 09/415,317

(22) Filed: Oct. 8, 1999

Related U.S. Application Data
(60) Provisional application No. 60/155,198, filed on Oct. 13, 1998.

(51) Int. Cl. C07J 1/00; A01N 45/00

(52) U.S. Cl. 514/26; 536/5

(57) ABSTRACT

This invention provides 5α-pregnane-3,β,(20S), 21-triol, 20-O-β-glucuronide and 5α-pregnane-3β,20R-diol, 20-O-β-glucuronide and pharmaceutically acceptable salts thereof which are useful as progestational agents.

This application claims the benefit of U.S. Provisional Application No. 60/155,198, which was converted from U.S. patent application Ser. No. 09/170,342, filed Oct. 13, 1998, pursuant to a petition filed under 37 C.F.R. 1.53(c)(2)(i).

### BACKGROUND OF THE INVENTION

The use of naturally occurring estrogenic compositions of substantial purity and low toxicity such as PREMARIN (conjugated equine estrogens) has become a preferred medical treatment for alleviating the symptoms of menopausal syndrome, osteoporosis/osteopenia in estrogen deficient women and in other hormone related disorders. The estrogenic components of the naturally occurring estrogenic compositions have been generally identified as sulfate esters of estrone, equilin, equilenin, 17-β-estradiol, dihydroequilenin and 17-β-dihydroequilenin (U.S. Pat. No. 2,834,712). The estrogenic compositions are usually buffered or stabilized with alkali metal salts of organic or inorganic acids at a substantially neutral pH of about 6.5 to 7.5. Urea has also been used as a stabilizer (U.S. Pat. No. 3,608,077). The incorporation of antioxidants to stabilize synthetic conjugated estrogens and the failure of pH control with tris(hydroxymethyl)aminomethane (TRIS) to prevent hydrolysis is discussed in U.S. Pat. No. 4,154,820.

Two of the compounds described herein, 5α-Pregnane-3β,(20S), 21 triol 20-O-β-glucuronide sodium salt and 5α-Pregnane-3β,20R-diol 20-O-β-glucuronide sodium salt are minor components of PREMARIN (conjugated equine estrogens).

### DESCRIPTION OF THE INVENTION

In accordance with this invention, there is provided 5α-Pregnane-3β,(20S), 21-triol, 20-O-β-glucuronide and 5α-Pregnane-3β,20R-diol, 20-O-β-glucuronide and pharmaceutically acceptable salts thereof which are useful as progestational agents. The preparation of pregnane glucuronides 7 and 12 is shown in schemes I and II, respectively. Additionally, a conversion of the glucuronides to the naturally occurring sodium salt forms 8 and 13 is also shown.

Pharmaceutically acceptable salts of 5α-Pregnane-3β,(20S), 21-triol 20-O-β-glucuronide and 5α-Pregnane-3β,20R-diol 20-O-β-glucuronide are not limited to the naturally occurring form, but also include the alkali metal salts, alkaline earth metal salts, ammonium salts, alkylammonium salts containing 1-6 carbon atoms or dialkylammonium salts containing 1-6 carbon atoms in each alkyl group, and trialkylammonium salts containing 1-6 carbon atoms in each alkyl group as well as any other atom or molecules which have a positive charge.

As 5α-Pregnane-3β,(20S), 21 triol 20-O-β-glucuronide and 5α-Pregnane-3β,20R-diol 20-O-β-glucuronide are minor components of PREMARIN (conjugated equine estrogens), this invention also provides 5α-Pregnane-3β,(20S), 21-triol 20-O-β -glucuronide and 5α-Pregnane-3β,20R-diol 20-O-β-glucuronide and their pharmaceutically acceptable salts in greater than 1 percent purity.

This invention also provides compounds consisting essentially of 5α-Pregnane-3β,(20S), 21-triol 20-O-β-glucuronide or a pharmaceutically acceptable salt thereof or 5α-Pregnane-3β,20R-diol 20-O-β-glucuronide or pharmaceutically acceptable salt thereof.

This invention further provides a method of using 5α-Pregnane-3β,(20S), 21-triol glucuronide and 5α-Pregnane-3β,20R-diol glucuronide or a pharmaceutically acceptable salt of the related glucuronides as progestational agents.

The starting materials used in this synthesis are either commercially available or can be prepared using standard chemical methodology.

The compounds of this invention can be prepared from readily available starting materials according to the processes in Scheme I, as shown for 5α-Pregnane-3β,(20S), 21-triol 20-O-β-glucuronide (and corresponding monobasic sodium salt) or according to the process in Scheme II, as shown for the synthesis of 5α-Pregnane-3β,20R-diol 20-O-β-glucuronide (and corresponding monobasic sodium salt).

In Scheme I, pregnenolone 3-acetate 1 was used as the starting material. Reaction of 1 with lead tetraacetate in acetic acid according to the procedure described by Purdy, et al, Journal of Medicinal Chemistry 33(6), 1572-1581 (1990) yields 2. Hydrogenation of 2 over PtO₂ in acetic acid yields compounds 3 and 4 in an approximate ratio of 3:1. Compound 3 is subsequently heated with excess equivalents of the acetobromo glucuronic acid methyl ester 5. The protected glucuronide is subsequently saponified with LiOH and reprotonated with acid to yield the desired 20-glucuronic acid conjugate 7. The free acid can be titrated with a slight excess of NaOH solution which generates the monosodium salt 8.

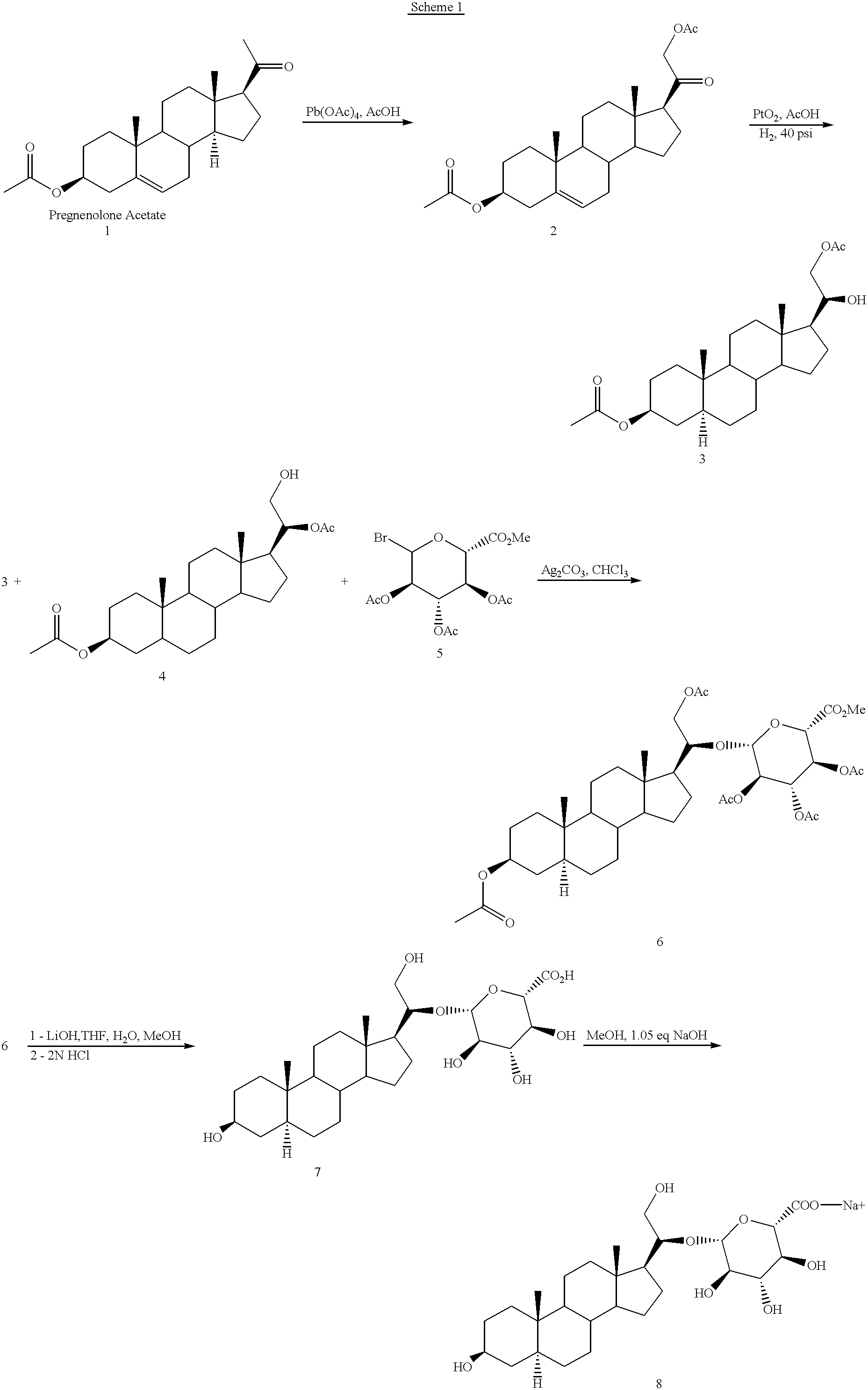

In Scheme II, compound 10 was reacted with acetobromo glucuronic acid methyl ester 5 and Ag₂CO₃ to yield the protected conjugate 11. This material was subsequently saponified and then protonated with acid to yield the deprotected glucuronic acid derivative 12. This material was then titrated with NaOH to yield the sodium glucuronate 13.

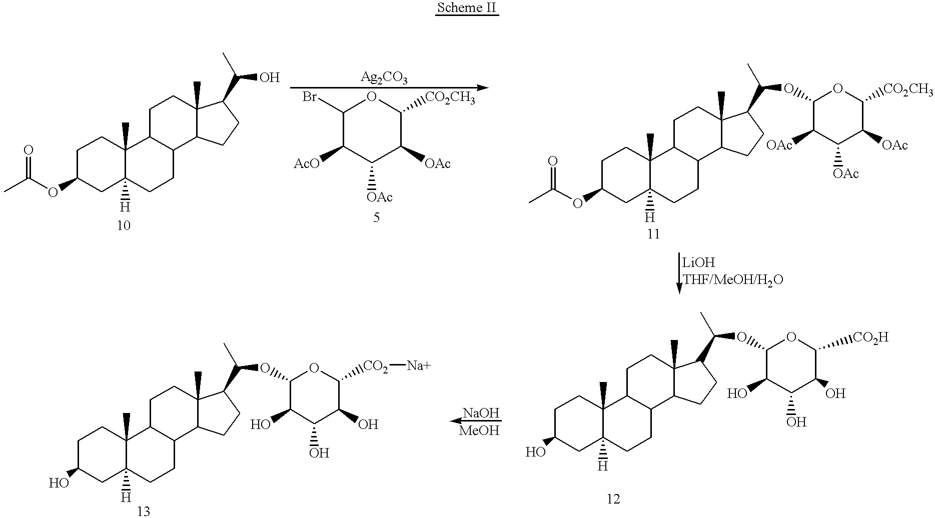

The compounds of this invention are protestional agents, and are therefore useful as oral contraceptives (male and female), in hormone replacement therapy (particularly when combined with an estrogen), in the treatment of endometriosis, luteal phase defects, benign breast and prostatic diseases and prostatic and endometrial cancers. The compounds of this invention are also useful in protecting against epileptic seizures, in cognition enhancement, in treating Alzheimer's disease, dementias, vasomotor symptoms related to menopause, and other central nervous system disorders The compounds of this invention are further useful in stimulating erythropoieses.

The compounds of this invention can be used alone as a sole therapeutic agent or can be used in combination with other agents, such as other estrogens, progestins, or androgens.

The compounds of this invention can be formulated neat or with a pharmaceutical carrier for administration, the proportion of which is determined by the solubility and chemical nature of the compound, chosen route of administration and standard pharmacological practice. The pharmaceutical carrier may be solid or liquid.

A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, lethicins, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellant.

Liquid pharmaceutical compositions which are sterile solutions or suspensions can be utilized by, for example, intramuscular, intraperitoneal or subcutaneous injection. Sterile solutions can also be administered intravenously. The compounds of this invention can also be administered orally either in liquid or solid composition form.

The compounds of this invention may be administered rectally or vaginally in the form of a conventional suppository. For administration by intranasal or intrabronchial inhalation or insufflation, the compounds of this invention may be formulated into an aqueous or partially aqueous solution, which can then be utilized in the form of an aerosol. The compounds of this invention may also be administered transdermally through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the active ingredient into the blood stream such as a semipermeable membrane covering a reservoir containing the active ingredient with or without a carrier, or a matrix containing the active ingredient. Other occlusive devices are known in the literature.

The dosage requirements vary with the particular compositions employed, the route of administration, the severity of the symptoms presented and the particular subject being treated. Based on the results obtained in the standard pharmacological test procedures, projected daily dosages of active compound would be 0.02 μg/kg-750 μg/kg. Treatment will generally be initiated with small dosages less than the optimum dose of the compound. Thereafter the dosage is increased until the optimum effect under the circumstances is reached; precise dosages for oral, parenteral, nasal, or intrabronchial administration will be determined by the administering physician based on experience with the individual subject treated. Preferably, the pharmaceutical composition is in unit dosage form, e.g. as tablets or capsules. In such form, the composition is sub-divided in unit dose containing appropriate quantities of the active ingredient; the unit dosage forms can be packaged compositions, for example, packaged powders, vials, ampoules, pre filled syringes or sachets containing liquids. The unit dosage form can be, for example, a capsule or tablet itself, or it can be the appropriate number of any such compositions in package form.

The following provides the preparation of representative compounds of this invention.

### EXAMPLE 1

### Pregnane-3β,20R,21-triol 3,21-diacetate 3

Compound 2 (6.0 g, 14.4 mmol) in 0.2 L AcOH was treated with PtO₂ (1.75 g, 7.7 mmol) and the solution was hydrogenated under 40 PSI of H₂. After 18 h, the catalyst was filtered and the crude reaction mixture was concentrated and chromatographed on silica gel using EtOAc/hexanes (1:4) to yield the two products (3 and 4). The first product to elute was the major (desired) material 3 which was isolated as 2.40 g of a white solid: Mp=164-166° C.; ¹H NMR (CDCl₃) 4.75-4.60 (m, 1 H), 4.16 (dd, 1 H, J=11.4 Hz, 2.2 Hz), 3.94-3.87 (m, 1 H),3.81-3.73 (m, 1 H), 2.10 (s, 3 H), 2.10-2.05 (m, 1 H), 2.02 (s, 3 H), 1.84 (d, 1 H, J=5.2 Hz), 1.82-0.88 (m, 21 H), 0.83 (s, 3 H), 0.76 (s, 3 H), 0.68 (dt, 1 H, J=10.6 Hz); MS (+ESI) 421 (M+H)⁺; IR (KBr) 3520, 2920, 2880, 1740, 1710 cm⁻¹.

### EXAMPLE 2

### Pregnane-3,β,20R,21-triol 3,20-diacetate 4

The second product 4 to elute was the minor product which was isolated as 0.73 g of a white solid: Mp=189-191° C.; ¹H NMR (DMSO) 4.91 (dq, 1 H, J=5.5 Hz, 2.2 Hz), 4.74-4.63 (m, 1 H), 3.81-3.74 (m, 1 H), 3.57-3.49 (m, 1 H), 2.09(s, 3 H), 2.02 (s, 3 H), 1.91 (dd, 1 H, J=7.2 Hz, 5.0 Hz), 1.85-0.83 (m, 22 H), 0.82(s, 3 H), 0.72-0.61 (m, 1 H), 0.65 (s, 3 H); MS EI 420 (M+); IR (KBr) 3410, 2920, 1730, 1700 cm⁻¹.

### EXAMPLE 3

### 2,3,4-O-Triacetyl-1-O-(3β,21-diacetoxy-5α-pregnan-20S-yl)-β-D-glucuronic acid methyl ester 6

Compound 3 (6.6 g, 15.7 mmol) was dissolved in CHCl₃ (125 mL) and treated with Ag₂CO₃ (4.3 g, 15.7 mmol) and the glucuronyl bromide 5 [21085-72-3] (6.2 g, 15.7 mmol). After refluxing for 1.5 h, an additional 0.5 eq of Ag₂CO₃ and 0.5 eq of the glucuronyl bromide were added. After an additional 1.5 h this addition of reagents was repeated and after another 1.5 h it was repeated once more. The reaction was allowed to reflux for a total of 5.5 h. The reaction was allowed to cool down and the inorganic salts were filtered off. The filtrate was concentrated and chromatographed on silica gel (3:7, EtOAc/hexanes) and the fractions containing product were concentrated and rechromatographed under the same conditions to yield 4.1 g of 6 which was triturated with MeOH to give 3.05 g of 6 as a white solid: Mp= 180-183° C.; MS (APCI) 754 (M+NH4⁺); ¹H NMR (DMSO) 5.36 (t, 1 H, J=9.6 Hz), 5.06 (d, 1 H, J=8.0 Hz), 4.91 (t, 1 H, J=9.8 Hz), 4.70 (dd, 1 H, J=9.5 Hz, 8.1 Hz), 4.63-4.50 (m, 1 H), 4.47 (d, 1 H, J=10.0 Hz), 4.16 (d, 1 H, J=11.3 Hz), 3.92-3.75 (m, 2 H), 3.63 (s, 3 H), 2.16-2.07 (m, 1 H), 2.04 (s, 3 H), 1.99 (s, 3 H), 1.97 (s, 3 H), 1.95 (s, 3 H), 1.93 (s, 3 H), 1.78-1.63 (m, 3 H), 1.62-1.39 (m, 6 H), 1.36-1.10 (m, 8H), 1.06-0.85 (m, 5 H), 0.79 (s, 3 H), 0.68 (s, 3 H).

### EXAMPLE 4

### 5α-Pregnane -3β,20S,21-triol 20-O-β-D-glucuronide 7

Compound 6 (3.76 g, 5.1 mmol) was dissolved in THF (25 mL) and treated with a solution consisting of LiOH (1.35 g, 56.1 mmol) in H₂O (13 mL). MeCH (4 mL) was added to this solution and the reaction was heated to 75° C. for 2 h. The solution was then cooled and concentrated. The aqueous residue was taken up with an additional 15 mL of H₂O. To this residue was then added a 2N HCl solution (43 mL). Solid formation was induced by scratching the glass. Filtration yielded 2.6 g of 7 as a white solid:

Mp=231-235° C.; ¹³C NMR (75 MHz, MeOD) (C═O missing), 102.6, 82.1, 77.6, 76.8, 75.3, 73.1, 71.9, 63.6, 57.6, 56.1, 51.5, 49.9, 46.3, 43.6, 40.3, 39.0, 38.3, 36.9, 36.7, 33.5, 32.2, 30.0, 25.9, 25.4, 22.3, 12.8, 11.9; ¹H NMR (300 MHz, MeOD) 4.54 (d, 1 H, J=7.7 Hz), 3.82-3.71 (m, 3 H), 3.59-3.22 (m, 5 H), 2.30 (d, 1 H, J=12.6 Hz), 1.75-1.66 (m, 6 H), 1.53-1.18 (m, 9 H), 1.17-0.88 (m, 6 H), 0.83 (s, 3 H), 0.78 (s, 3 H), 0.70-0.58 (m, 1 H); MS (−)ESI 511 (M−H)⁻; IR (KBr) 3410, 2910, 2830, 1730, 1700 cm⁻¹.

### EXAMPLE 5

### 5α-Pregnane-3β,20S,21-triol 20-O-β-D-glucuronide sodium salt 8

Compound 7 (1.57 g, 1.7 mmol) was suspended in MeOH (20 mL) and treated with an aqueous solution of NaOH (6.4 mL, 0.5 N) and stirred for 5 minutes which allowed all of the starting material to go into solution. The solution was then mixed with 0.65 g of product from a previous run and the combination stripped onto silica gel and column chromatographed on silica gel (MeOH:CH₂Cl₂, 4:6 then 5:5). The product was then triturated once with a 1:1 mixture of MeOH and Dioxane (total volume equal 60 mL) to yield 1.57 g of 8 as a white solid: Mp=240-244° C.; ¹³C NMR (75 MHz, DMSO) (C═O missing), 100.4, 79.8, 76.7, 73.9, 73.7, 72.2, 69.3, 66.3, 62.0, 55.6, 54.0, 49.8, 44.3, 41.9, 38.1, 36.6, 35.1, 35.0, 31.8, 31.3, 28.4, 24.4, 23.9, 20.8, 12.1, 11.4; IR (KBr) 3400 (H₂O), 2920, 2870, 1610 cm⁻¹.

### EXAMPLE 6

### Pregnane-3β,20S,21-triol 9

Compound 3 (1.6 g, 3.8 mmol) was dissolved in TBF (8 mL). A solution of LiOH (0.27 g, 11.4 mmol) in 3 mL of H₂O was added. In order to make the solution one phase, 1 mL of MeOH was added to the reaction mixture. After 30 minutes at reflux, the reaction mixture was treated with 1.1 mL of AcOH and partitioned between an organic layer consisting of 100 mL EtOAc, 20 mL CH₂Cl₂, 10 mL MeOH, and 60 mL H₂O. The organic layer was then washed with brine and dried over MgSO₄. The solution was concentrated and the resulting solid triturated with ether to yield 1.1 g of 9 as a white solid: Mp=210-212° C.; ¹H NMR (DMSO) (3 OH protons missing) 4.43 (d, 1 H, J=4.6 Hz), 4.31 (t, 1 H, J=5.5 Hz), 4.05 (d, 1 H, J=5.2 Hz), 3.16-3.05 (m, 1 H), 2.10 (d, 1 H, J=12.4 Hz), 1.65-0.78 (m, 21 H), 0.75 (s, 3 H), 0.68 (s, 3 H), 0.65-0.54 (m, 1 H); MS EI 336 M+; IR (KBr) 3400, 2930, 2880 cm⁻¹.

### EXAMPLE 7

### 1-O-(3β-Acetoxy-pregn-20-yl)-2,3,4-triacetyl-β-D-glucuronic acid methyl ester 11

5α-Pregnan-3β, 20β-diol 3-acetate 10 (3.0 g, 11.0 mmol), glucuronyl bromide 5 [21085-72-3] (5.5 g, 13.8 mmol) and Ag₂CO₃ (4.5 g, 16.1 mmol) were stirred in toluene (40 mL) at rt (flask protected from light with aluminum foil). After 18 h the reaction was diluted with CHCl₂, filtered, concentrated and chromatographed on silica gel (EtOAc:Hex, 2:8 then EtOAc:Hex, 4:6) to yield a solid which was triturated with MeOH to give 3.0 g of 11 as a white solid:

Mp=248-251° C.; ¹H NMR (DMSO) 5.33 (t, 1 H, J=9.6 Hz), 4.95-4.88 (m, 2 H), 4.68 (dd, 1 H, J=9.5 Hz, 8.1 Hz), 4.62-4.49 (m, 1 H), 4.44 (d, 1 H, J=10.0 Hz), 3.71-3.65 (m, 1 H), 3.63 (s, 3 H), 2.11 (d, 1 H, J=12.6 Hz), 1.98 (s, 3 H), 1.97 (s, 3 H), 1.96 (s, 3 H), 1.95 (s, 3 H), 1.77-1.64 (m, 2 H), 1.63-1.10 (m, 14 H), 0.99 (d, 3 H, J=5.8 Hz), 0.95-0.83 (m, 5 H), 0.78 (s, 3 H), 0.66 (s, 3 H), 0.65-0.58 (m, 1 H); IR (KBr) 2930, 2900, 2830, 1750, 1730 cm⁻¹; MS (+)ESI 696 (M+NH₄)⁺.

### EXAMPLE 8

### 5α-Pregnane-3β,20R-diol 20-O-β-D-glucuronide 12

Compound 11 (2.5 g, 3.7 mmol) was dissolved in a solution of THF (25 mL) and MeOH (15 mL) and treated with a solution of LiOH (0.9 g, 37.5 mmol) in H₂O (10 mL). The reaction was heated at reflux for 1 h. The reaction was allowed to cool to room temperature and concentrated. The product was taken up in water (20 mL) and acidified with aqueous 2 N HCl. A precipitate formed which was filtered yielding 1.5 g of 12 as a white solid: Mp=255-260°; ¹H NMR (DMSO) (3 H buried) 4.98 (d, 1 H, J=3.8 Hz), 4.91 (d, 1 H, J=4.6 Hz), 4.42 (br s, 1 H), 4.25 (d, 1 H, J=7.7 Hz), 3.71-3.62 (m, 1 H), 3.58 (d, 1 H, J=9.7 Hz), 3.23-3.12 (m, 1 H), 2.96-2.87 (m, 1 H), 2.15 (d, 1 H, J=12.3 Hz), 1.67-1.46 (m, 5 H), 1.45-0.80 (m, 17 H), 1.01 (d, 3 H, J=5.8 Hz), 0.74 (s, 3 H), 0.65 (s, 3 H), 0.65-0.52 (m, 1 H); IR (KBr) 3520, 3400, 2920, 2820, 2800, 1710 cm⁻¹; MS (−) ESI 495 (M−H)⁻.

### EXAMPLE 9

### 5α-Pregnane-3β,20R-diol 20-O-β-D-glucuronide sodium salt 13

A suspension of Compound 12 (1.5 g, 3.0 mmol) in MeOH (25 mL) and treated with an aqueous solution of NaOH (0.5 N, 6.0 mL, 3.0 mmol) and stirred for 5 minutes during which time everything went into solution. The solution was concentrated to dryness and the residue was triturated with EtOH to obtain 1.1 g of 13 as a white solid:

Mp=223-226° C. (dec); ¹H NMR (DMSO) 7.26 (s, 1 H), 4.76 (d, 1 H, J=3.6 Hz), 4.67 (d, 1 H, J=3.6 Hz), 4.43 (d, 1 H, J=approx 4 Hz), 4.12 (d, 1 H, J=7.6 Hz), 3.78-3.71 (m, 1 H), 3.45-3.35 (m, 1 H), 3.17-3.03 (m, 3 H), 3.38-3.29 (m, 1 H), 2.17 (d, 1 H, J=12.4 Hz), 1.67-1.49 (m, 5 H), 1.45-0.80 (m, 17 H), 0.99 (d, 3 H, J= 5.9 Hz), 0.76 (s, 3 H), 0.75 (s, 3 H), 0.65-0.53 (m, 1 H); IR (KBr) 3400, 2920, 2830, 1610 cm⁻¹; MS (−) ESI 495 (M−H)⁻.

What is claimed is:

1. A compound which is 5α-pregnane-3β,20S,21-triol 20-O-β-D-glucuronide or a pharmaceutically acceptable salt thereof.

2. The compound of claim 1, wherein the wherein the pharmaceutically acceptable salt of the 20-glucuronide is an alkali metal salt, alkaline earth metal salt, ammonium salt, alkylammonium salt containing 1-6 carbon atoms, or dialkylammonium salt containing 1-6 carbon atoms in each alkyl group, or trialkylammonium salt containing 1-6 carbon atoms in each alkyl group.

3. A compound which is 5α-pregnane-3β,20R-diol 20-O-β-D-glucuronide or a pharmaceutically acceptable salt thereof.

4. The compounds of claim 3 wherein the pharmaceutically acceptable salt of the 20-glucuronide is an alkali metal salt, alkaline earth metal salt, ammonium salt, alkylammonium salt containing 1-6 carbon atoms, or dialkylammonium salt containing 1-6 carbon atoms in each alkyl group, or trialkylammonium salt containing 1-6 carbon atoms in each alkyl group.

5. 5α-pregnane-3β,20S,21-triol 20-O-β-D-glucuronide or a pharmaceutically acceptable salt thereof, which is at least 1 percent pure.

6. 5α-pregnane-3β,20R-diol 20-O-β-D-glucuronide or a pharmaceutically acceptable salt thereof which is at least 1 percent pure.

7. A compound consisting essentially of 5α-pregnane -3β,20S,21-triol 20-O-β-D-glucuronide or a pharmaceutically acceptable salt thereof.

8. A compound consisting essentially of 5α-pregnane-3β,20R-diol 20-O-β-D-glucuronide or a pharmaceutically acceptable salt thereof.

9. A compound consisting essentially of 5α-pregnane-3β,20S,21-triol 20-O-β-D-glucuronide sodium salt.

10. A compound consisting essentially of 5α-pregnane-3β,20R-diol 20-O-β-D-glucuronide sodium salt.

11. A pharmaceutical composition which comprises 5α-pregnane-3β,20S,21-triol 20-O-β-D-glucuronide or a pharmaceutically acceptable salt thereof and a pharmaceutical carrier.

12. A pharmaceutical composition which comprises 5α-pregnane-3β,20R-diol 20-O-β -D-glucuronide or a pharmaceutically acceptable salt thereof and a pharmaceutical carrier.

13. A method of providing progestational therapy to a mammal in need thereof which comprises administering a progestationally effective amount of 5α-pregnane-3β,20S,21-triol 20-O-β-D-glucuronide or a pharmaceutically acceptable salt thereof to said mammal.

14. A method of providing progestational therapy to a mammal in need thereof which comprises administering a progestationally effective amount of 5α-pregnane-3β,20R-diol 20-O-β-D-glucuronide or a pharmaceutically acceptable salt thereof to said mammal.

15. A method of treating or inhibiting cancers, central nervous system disorders, dementias, or alzheimer's disease in a mammal in need thereof, which comprises administering an effective amount of 5α-pregnane-3β,20S,21-triol 20-O-β-D-glucuronide or a pharmaceutically acceptable salt thereof, to said mammal.

16. A method of treating or inhibiting cancers, central nervous system disorders, dementias, or alzheimer's disease in a mammal in need thereof, which comprises administering an effective amount of 5α-pregnane-3β,20R-diol 20-O-β-D-glucuronide or a pharmaceutically acceptable salt thereof, to said mammal.
